(19) United States
(12) Patent Application Publication
(10) Pub. No.: US 2010/0273871 A1
(43) Pub. Date: Oct. 28, 2010

(54) ANTICANCER TOCOPHERYL SUCCINATE DERIVATIVES

(75) Inventors: Ching-Shih Chen, Upper Arlington, OH (US); Dasheng Wang, Dublin, OH (US)

Correspondence Address: CALFEE HALTER & GRISWOLD, LLP, 800 SUPERIOR AVENUE, SUITE 1400, CLEVELAND, OH, 44114, US

(73) Assignee: THE OHIO STATE UNIVERSITY RESEARCH FOUNDATION, Columbus, OH (US)

(21) Appl. No.: 12/768,392

(22) Filed: Apr. 27, 2010

Related U.S. Application Data
(63) Continuation of application No. 11/708,792, filed on Feb. 21, 2007, now abandoned.
(60) Provisional application No. 60/775,107, filed on Feb. 21, 2006.

(51) Int. Cl. A61K 31/355; C07D 311/72; A61P 35/00

(52) U.S. Cl. 514/458; 549/410; 549/408

(57) ABSTRACT

Compounds of formula I:

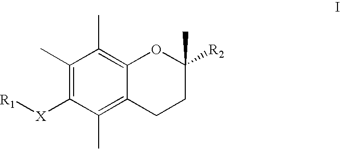

wherein X is selected from oxygen, nitrogen and sulfur; n is 0 or 1; R₁ is selected from alkyl, carboxylic acid, carboxylate, carboxamide, ester and combinations thereof; R₂ is selected from alkyl, substituted alkyl, carboxylic acid, carboxylate, carboxamide, sulfonyl, sulfonamide and combinations thereof; and derivatives and metabolites thereof. Further provided are methods of using a compound of formula I to prevent and/or treat a subject having a condition characterized by unwanted cell proliferation. Also provided are pharmaceutical compounds comprising one or more compounds of formula I, or derivatives or pharmaceutically acceptable salts thereof.

### CROSS-REFERENCE TO RELATED APPLICATIONS

This application claims priority to, and any benefit of, U.S. Provisional Patent Application Ser. No. 60/775,107, entitled “ANTICANCER AGENTS” filed Feb. 21, 2006, the entirety of which is incorporated herein by reference.

### STATEMENT ON FEDERALLY FUNDED RESEARCH

This invention was funded, at least in part, by National Institutes of Health Grant CA-112250 and Department of Defense Prostate Cancer Research Program Award W81XWH-05-1-0089. The federal government may have certain rights in this invention.

### BACKGROUND OF THE INVENTION

Recent investigations have suggested the potential use of α-tocopheryl succinate as a cancer therapeutic agent. Evidence indicates that alpha-tocopheryl succinate induces apoptosis in cells with a malignant or transformed phenotype without incurring significant toxicity to normal cells. Moreover, its in vivo efficacy has been demonstrated in a number of animal model experiments, including suppression of breast and melanoma tumor growth, inhibition of colon cancer liver metastases, and sensitization of colon tumor cells to the tumor necrosis factor-related apoptosis-inducing ligand (TRAIL). Despite these advances, the mechanism underlying the effect of this redox-inactive vitamin E derivative on apoptosis remains elusive.

A need exists for new anticancer agents that can induce apoptosis in cancer cells without incurring significant toxicity to normal cells. One approach to finding new anticancer agents is to determine one or more major targets by which alpha-tocopheryl succinate mediated antineoplastic activities in prostate cancer cells and then develop pharmaceutical agents.

### SUMMARY OF THE INVENTION

Provided herein are the compounds of formula I:

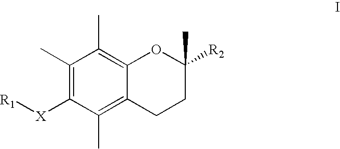

wherein X is selected from the group consisting of oxygen, nitrogen and sulfur; R₁ is selected from the group consisting of hydrogen, alkyl, carboxylic acid, carboxylate, carboxamide, ester and combinations thereof; R₂ is selected from the group consisting of alkyl, substituted alkyl, carboxylic acid, carboxylate, carboxamide, sulfonyl, sulfonamide and combinations thereof; and derivatives and metabolites thereof.

Also provided are prevention and/or treatment of a cell proliferative disease comprising in a subject by administering to the subject a pharmacologically effective dose of a compound of formula I. Also provided are pharmaceutical compositions comprising one or more compounds of formula I.

### BRIEF DESCRIPTION OF THE DRAWINGS

FIG. 1 shows a first synthetic scheme for preparing the compounds described herein.

FIG. 2 shows a second synthetic scheme for preparing the compounds described herein.

FIG. 3 shows a third synthetic scheme for preparing the compounds described herein.

FIG. 4 shows differential sensitivity of PC-3, LNCaP, and Bcl-xL-overexpressing LNCaP (LNCaP/B3) cells to α-tocopheryl succinate-induced apoptosis.

FIG. 5 shows α-Tocopheryl succinate blocks Bcl-xL/Bcl-2 function by inhibiting BH3 domain-mediated heterodimerization.

FIG. 6 shows modeled docking of α-tocopheryl succinate (upper panel) and TS-1 into the Bak BH3 peptide-binding site of Bcl-xL.

FIG. 7 shows structures and potency for inhibiting Bak BH3 peptide binding to Bcl-xL and for suppressing the viability of PC-3 and LNCaP cells for α-tocopheryl succinate and TS-1-TS-5.

FIG. 8 shows mechanistic validation of the antitumor action of TS-1. (A) Evidence of apoptotic death in drug-treated PC-3 cells.

### DETAILED DESCRIPTION OF THE INVENTION

Provided herein are the compounds of formula I:

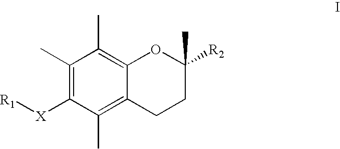

wherein X is selected from the group consisting of oxygen, nitrogen and sulfur; R₁ is selected from the group consisting of hydrogen, alkyl, carboxylic acid, carboxylate, carboxamide, ester and combinations thereof; R₂ is selected from the group consisting of alkyl, substituted alkyl, carboxylic acid, carboxylate, carboxamide, sulfonyl, sulfonamide and combinations thereof; and derivatives and metabolites thereof.

In some specific embodiments, the compounds of formula I are selected from 2,5,7,8-tetramethyl-(2R-(4-methylpentyl)chroman-6-acetic acid, 2,5,7,8-tetramethyl-(2R-(4-methylpentyl)chroman-6-propionic acid, 2,5,7,8-tetramethyl-(2R-(4-methylpentyl)chroman-6-butyric acid, 2,5,7,8-tetramethyl-(2R-(4,8-dimethylnonanyl)chroman-6-acetic acid, 2,5,7,8-tetramethyl-(2R-(4,8-dimethylnonanyl)chroman-6-propionic acid, 2,5,7,8-tetramethyl-(2R-(4,8-dimethylnonanyl)chroman-6-butyric acid, 2,5,7,8-tetramethyl-(2R-(4-methylpentyl)chroman-6-succinate, 2,5,7,8-tetramethyl-(2R-(4-methylpentyl)chroman-6-glutarate, 2,5,7,8-tetramethyl-(2R-(4,8-dimethylnonanyl)chroman-6-succinate, 2,5,7,8-tetramethyl-(2R-(4,8-dimethylnonanyl)chroman-6-glutarate, 6-hydroxy-2,5,7,8-tetramethyl-(2R-(4-cyanobutyl)chroman, 6-hydroxy-2,5,7,8-tetramethyl-(2R-(7-cyanoheptyl)chroman, 6-hydroxy-2,5,7,8-tetramethyl-(2R-(9-cyanononyl)chroman, 6-hydroxy-2,5,7,8-tetramethyl-(2R-(5-aminopentyl)chroman, 6-hydroxy-2,5,7,8-tetramethyl-(2R-(8-aminooctyl)chroman, 6-hydroxy-2,5,7,8-tetramethyl-(2R-(10-aminodecyl)chroman, 6-hydroxy-2,5,7,8-tetramethyl-(2R-(5-methylsulfonamidepentyl)chroman, 6-hydroxy-2,5,7,8-tetramethyl-(2R-(8-methylsulfonamideoctyl)chroman, 6-hydroxy-2,5,7,8-tetramethyl-(2R-(10-methylsulfonamidedecyl)chroman, 6-hydroxy-2,5,7,8-tetramethyl-(2R-(5-aminosulfonamidepentyl)chroman, 6-hydroxy-2,5,7,8-tetramethyl-(2R-(8-aminosulfonamideoctyl)chroman, 6-hydroxy-2,5,7,8-tetramethyl-(2R-(10-aminosulfonamidedecyl)chroman, 6-succinate-2,5,7,8-tetramethyl-(2R-(4-cyanobutyl)chroman, 6-succinate-2,5,7,8-tetramethyl-(2R-(7-cyanoheptyl)chroman, 6-succinate-2,5,7,8-tetramethyl-(2R-(9-cyanononyl)chroman, 6-succinate-2,5,7,8-tetramethyl-(2R-(5-aminopentyl)chroman, 6-succinate-2,5,7,8-tetramethyl-(2R-(8-aminooctyl)chroman, 6-succinate-2,5,7,8-tetramethyl-(2R-(10-aminodecyl)chroman, 6-succinate-2,5,7,8-tetramethyl-(2R-(5-methylsulfonamidepentyl)chroman, 6-succinate-2,5,7,8-tetramethyl-(2R-(8-methylsulfonamideoctyl)chroman, 6-succinate-2,5,7,8-tetramethyl-(2R-(10-methylsulfonamidedecyl)chroman, 6-succinate-2,5,7,8-tetramethyl-(2R-(5-aminosulfonamidepentyl)chroman, 6-succinate-2,5,7,8-tetramethyl-(2R-(8-aminosulfonamideoctyl)chroman, 6-succinate-2,5,7,8-tetramethyl-(2R-(10-aminosulfonamidedecyl)chroman and derivatives and metabolites thereof.

Further provided are methods for the prevention and/or treatment of a cell proliferative disease comprising administering to an animal a pharmacologically effective dose of a compound of formula I:

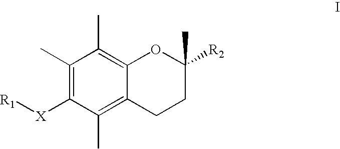

wherein X is selected from the group consisting of oxygen, nitrogen and sulfur; R₁ is selected from the group consisting of hydrogen, alkyl, carboxylic acid, carboxylate, carboxamide, ester and combinations thereof; R₂ is selected from the group consisting of alkyl, substituted alkyl, carboxylic acid, carboxylate, carboxamide, sulfonyl, sulfonamide and combinations thereof; and derivatives and metabolites thereof. In an exemplary embodiment, X is O, and X—R₁ is either hydroxy or carboxylic acid.

In some specific embodiments, the compound of formula I is selected from 2,5,7,8-tetramethyl-(2R-(4-methylpentyl)chroman-6-acetic acid, 2,5,7,8-tetramethyl-(2R-(4-methylpentyl)chroman-6-propionic acid, 2,5,7,8-tetramethyl-(2R-(4-methylpentyl)chroman-6-butyric acid, 2,5,7,8-tetramethyl-(2R-(4,8-dimethylnonanyl)chroman-6-acetic acid, 2,5,7,8-tetramethyl-(2R-(4,8-dimethylnonanyl)chroman-6-propionic acid, 2,5,7,8-tetramethyl-(2R-(4,8-dimethylnonanyl)chroman-6-butyric acid, 2,5,7,8-tetramethyl-(2R-(4-methylpentyl)chroman-6-succinate, 2,5,7,8-tetramethyl-(2R-(4-methylpentyl)chroman-6-glutarate, 2,5,7,8-tetramethyl-(2R-(4,8-dimethylnonanyl)chroman-6-succinate, 2,5,7,8-tetramethyl-(2R-(4,8-dimethylnonanyl)chroman-6-glutarate, 6-hydroxy-2,5,7,8-tetramethyl-(2R-(4-cyanobutyl)chroman, 6-hydroxy-2,5,7,8-tetramethyl-(2R-(7-cyanoheptyl)chroman, 6-hydroxy-2,5,7,8-tetramethyl-(2R-(9-cyanononyl)chroman, 6-hydroxy-2,5,7,8-tetramethyl-(2R-(5-aminopentyl)chroman, 6-hydroxy-2,5,7,8-tetramethyl-(2R-(8-aminooctyl)chroman, 6-hydroxy-2,5,7,8-tetramethyl-(2R-(10-aminodecyl)chroman, 6-hydroxy-2,5,7,8-tetramethyl-(2R-(5-methylsulfonamidepentyl)chroman, 6-hydroxy-2,5,7,8-tetramethyl-(2R-(8-methylsulfonamideoctyl)chroman, 6-hydroxy-2,5,7,8-tetramethyl-(2R-(10-methylsulfonamidedecyl)chroman, 6-hydroxy-2,5,7,8-tetramethyl-(2R-(5-aminosulfonamidepentyl)chroman, 6-hydroxy-2,5,7,8-tetramethyl-(2R-(8-aminosulfonamideoctyl)chroman, 6-hydroxy-2,5,7,8-tetramethyl-(2R-(10-aminosulfonamidedecyl)chroman, 6-succinate-2,5,7,8-tetramethyl-(2R-(4-cyanobutyl)chroman, 6-succinate-2,5,7,8-tetramethyl-(2R-(7-cyanoheptyl)chroman, 6-succinate-2,5,7,8-tetramethyl-(2R-(9-cyanononyl)chroman, 6-succinate-2,5,7,8-tetramethyl-(2R-(5-aminopentyl)chroman, 6-succinate-2,5,7,8-tetramethyl-(2R-(8-aminooctyl)chroman, 6-succinate-2,5,7,8-tetramethyl-(2R-(10-aminodecyl)chroman, 6-succinate-2,5,7,8-tetramethyl-(2R-(5-methylsulfonamidepentyl)chroman, 6-succinate-2,5,7,8-tetramethyl-(2R-(8-methylsulfonamideoctyl)chroman, 6-succinate-2,5,7,8-tetramethyl-(2R-(10-methylsulfonamidedecyl)chroman, 6-succinate-2,5,7,8-tetramethyl-(2R-(5-aminosulfonamidepentyl)chroman, 6-succinate-2,5,7,8-tetramethyl-(2R-(8-aminosulfonamideoctyl)chroman, 6-succinate-2,5,7,8-tetramethyl-(2R-(10-aminosulfonamidedecyl)chroman and derivatives and metabolites thereof.

In accordance with the methods described herein, the compounds of formula I generally exhibit an anti-proliferative effect including, but not limited to one or more of apoptosis, cell cycle arrest, cellular differentiation, or DNA synthesis arrest. The methods disclosed herein are especially suitable for use in humans.

Further provided is a pharmaceutical composition including one or more compounds of formula I and a pharmaceutical carrier. In some specific embodiments, the pharmaceutical composition comprises one or more of the following compounds of formula I: 2,5,7,8-tetramethyl-(2R-(4-methylpentyl)chroman-6-acetic acid, 2,5,7,8-tetramethyl-(2R-(4-methylpentyl)chroman-6-propionic acid, 2,5,7,8-tetramethyl-(2R-(4-methylpentyl)chroman-6-butyric acid, 2,5,7,8-tetramethyl-(2R-(4,8-dimethylnonanyl)chroman-6-acetic acid, 2,5,7,8-tetramethyl-(2R-(4,8-dimethylnonanyl)chroman-6-propionic acid, 2,5,7,8-tetramethyl-(2R-(4,8-dimethylnonanyl)chroman-6-butyric acid, 2,5,7,8-tetramethyl-(2R-(4-methylpentyl)chroman-6-succinate, 2,5,7,8-tetramethyl-(2R-(4-methylpentyl)chroman-6-glutarate, 2,5,7,8-tetramethyl-(2R-(4,8-dimethylnonanyl)chroman-6-succinate, 2,5,7,8-tetramethyl-(2R-(4,8-dimethylnonanyl)chroman-6-glutarate, and 2 carboxamidebutyl)chroman-6-butyric acid, 6-hydroxy-2,5,7,8-tetramethyl-(2R-(4-cyanobutyl)chroman, 6-hydroxy-2,5,7,8-tetramethyl-(2R-(7-cyanoheptyl)chroman, 6-hydroxy-2,5,7,8-tetramethyl-(2R-(9-cyanononyl)chroman, 6-hydroxy-2,5,7,8-tetramethyl-(2R-(5-aminopentyl)chroman, 6-hydroxy-2,5,7,8-tetramethyl-(2R-(8-aminooctyl)chroman, 6-hydroxy-2,5,7,8-tetramethyl-(2R-(10-aminodecyl)chroman, 6-hydroxy-2,5,7,8-tetramethyl-(2R-(5-methylsulfonamidepentyl)chroman, 6-hydroxy-2,5,7,8-tetramethyl-(2R-(8-methylsulfonamideoctyl)chroman, 6-hydroxy-2,5,7,8-tetramethyl-(2R-(10-methylsulfonamidedecyl)chroman, 6-hydroxy-2,5,7,8-tetramethyl-(2R-(5-aminosulfonamidepentyl)chroman, 6-hydroxy-2,5,7,8-tetramethyl-(2R-(8-aminosulfonamideoctyl)chroman, 6-hydroxy-2,5,7,8-tetramethyl-(2R-(10-aminosulfonamidedecyl)chroman, 6-succinate-2,5,7,8-tetramethyl-(2R-(4-cyanobutyl)chroman, 6-succinate-2,5,7,8-tetramethyl-(2R-(7-cyanoheptyl)chroman, 6-succinate-2,5,7,8-tetramethyl-(2R-(9-cyanononyl)chroman, 6-succinate-2,5,7,8-tetramethyl-(2R-(5-aminopentyl)chroman, 6-succinate-2,5,7,8-tetramethyl-(2R-(8-aminooctyl)chroman, 6-succinate-2,5,7,8-tetramethyl-(2R-(10-aminodecyl)chroman, 6-succinate-2,5,7,8-tetramethyl-(2R-(5-methylsulfonamidepentyl)chroman, 6-succinate-2,5,7,8-tetramethyl-(2R-(8-methylsulfonamideoctyl)chroman, 6-succinate-2,5,7,8-tetramethyl-(2R-(10-methylsulfonamidedecyl)chroman, 6-succinate-2,5,7,8-tetramethyl-(2R-(5-aminosulfonamidepentyl)chroman, 6-succinate-2,5,7,8-tetramethyl-(2R-(8-aminosulfonamideoctyl)chroman, 6-succinate-2,5,7,8-tetramethyl-(2R-(10-aminosulfonamidedecyl)chroman.

In one exemplary embodiment, the pharmaceutical composition includes a therapeutically effective amount of one or more of the compounds of formula I in association with an acceptable carrier. In another exemplary embodiment, the pharmaceutical composition includes a therapeutically effective amount of one or more of the compounds of formula I in association with an acceptable carrier and one or more adjuvants. In another exemplary embodiment, the pharmaceutical composition includes a therapeutically effective amount of one or more of the compounds of formula I in association with an acceptable carrier, one or more adjuvants and one or more diluents. In any of these exemplary embodiments, one or more of the compounds of formula I may be pharmaceutically acceptable salts thereof. In any of these exemplary embodiments one or more of the compounds of formula I may be derivatives of formula I.

The compounds and methods of the present invention are useful for, but not limited to treating, inhibiting, or delaying the onset of cancers. The compounds and methods are also useful in the treatment of precancers and other incidents of undesirable cell proliferation. According to the present invention, the compounds of formula I are administered to a subject experiencing undesirable cell proliferation. The compounds and methods are useful for treating cancers including, but not limited to, leukemia, non-small cell lung cancer, colon cancer, CNS cancer, melanoma, ovarian cancer, renal cancer, prostate cancer, bladder cancer, lymphoma, and breast cancer. Furthermore, they are useful in the prevention of these cancers in individuals with precancers, as well as individuals prone to these disorders.

The term “treatment” includes partial or total destruction of the undesirable proliferating cells with minimal destructive effects on normal cells. In accordance with the present invention, desired mechanisms of treatment at the cellular include, but are not limited to one or more of apoptosis, cell cycle arrest, cellular differentiation, or DNA synthesis arrest.

The term “prevention” includes either preventing the onset of a clinically evident unwanted cell proliferation altogether or preventing the onset of a preclinically evident stage of unwanted rapid cell proliferation in individuals at risk. Also intended to be encompassed by this definition is the prevention of metastasis of malignant cells or to arrest or reverse the progression of malignant cells. This includes prophylactic treatment of those at risk of developing precancers and cancers.

The terms “therapeutically effective” and “pharmacologically effective” are intended to qualify the amount of each agent which will achieve the goal of improvement in disease severity and the frequency of incidence, while avoiding adverse side effects typically associated with alternative therapies.

The term “subject” for purposes of treatment includes any human or animal subject who has a disorder characterized by unwanted, rapid cell proliferation. Such disorders include, but are not limited to cancers and precancers. For methods of prevention the subject is any human or animal subject, and preferably is a human subject who is at risk of acquiring a disorder characterized by unwanted, rapid cell proliferation, such as cancer. The subject may be at risk due to exposure to carcinogenic agents, being genetically predisposed to disorders characterized by unwanted, rapid cell proliferation, and so on. Besides being useful for human treatment, the compounds of the present invention are also useful for veterinary treatment of mammals, including companion animals and farm animals, such as, but not limited to dogs, cats, horses, cows, sheep, and pigs. Preferably, subject means a human.

The terms “proliferative cells,” “proliferating cells,” “rapidly proliferating cells,” “undesirable proliferating cells,” “undesirable rapidly proliferating cells,” “unwanted rapidly proliferating cells,” and the like, refer to cancer cells, precancer cells, and other abnormal, rapidly dividing cells in a subject.

“Derivatives” as used herein, is intended to encompass any compounds which are structurally related to the compounds of formula I which possess substantially equivalent activity, as measured by the derivative's ability to induce apoptosis, cell cycle arrest, cellular differentiation, or DNA synthesis arrest. By way of example, such compounds may include, but are not limited to salts, esters, metabolites, and prodrugs thereof. Such compounds may be formed in vivo, such as by metabolic mechanisms.

Where the term alkyl is used, either alone or with other terms, such as haloalkyl or alkylaryl, it includes C₁ to C₁₀ linear or branched alkyl radicals, examples include methyl, ethyl, propyl, isopropyl, butyl, tent-butyl, and so forth. The term “haloalkyl” includes C₁ to C₁₀ linear or branched alkyl radicals substituted with one or more halo radicals. Some examples of haloalkyl radicals include trifluoromethyl, 1,2-dichloroethyl, 3-bromopropyl, and so forth. The term “halo” includes radicals selected from F, Cl, Br, and I. Alkyl radical substituents of the present invention may also be substituted with other groups such as azido, for example, azidomethyl, 2-azidoethyl, 3-azidopropyl and so on.

The term aryl, used alone or in combination with other terms such as alkylaryl, haloaryl, or haloalkylaryl, includes such aromatic radicals as phenyl, biphenyl, and benzyl, as well as fused aryl radicals such as naphthyl, anthryl, phenanthrenyl, fluorenyl, and indenyl and so forth. The term “aryl” also encompasses “heteroaryls,” which are aryls that have carbon and one or more heteroatoms, such as O, N, or S in the aromatic ring. Examples of heteroaryls include indolyl, pyrrolyl, and so on. “Alkylaryl” or “arylalkyl” refers to alkyl-substituted aryl groups such as butylphenyl, propylphenyl, ethylphenyl, methylphenyl, 3,5-dimethylphenyl, tert-butylphenyl and so forth. “Haloaryl” refers to aryl radicals in which one or more substitutable positions has been substituted with a halo radical, examples include fluorophenyl, 4-chlorophenyl, 2,5-chlorophenyl and so forth. “Haloalkylaryl” refers to aryl radicals that have a haloalkyl substituent. Examples of haloalkylaryls include such radicals as bromomethylphenyl, 4-bromobutylphenyl and so on. Carboxyamide refers to the group —CONH₂, and sulfonamide refers to the group —SO₂NH₂.

Also included in the family of compounds of formula I are the pharmaceutically acceptable salts thereof. The phrase “pharmaceutically acceptable salts” connotes salts commonly used to form alkali metal salts and to form addition salts of free acids or free bases. The nature of the salt is not critical, provided that it is pharmaceutically acceptable. Suitable pharmaceutically acceptable acid addition salts of compounds of formula I may be prepared from an inorganic acid or from an organic acid. Examples of such inorganic acids are hydrochloric, hydrobromic, hydroiodic, nitric, carbonic, sulfuric, and phosphoric acid. Appropriate organic acids may be selected from aliphatic, cycloaliphatic, aromatic, araliphatic, heterocyclic, carboxylic, and sulfonic classes of organic acids, examples of which include formic, acetic, propionic, succinic, glycolic, gluconic, lactic, malic, tartaric, citric, ascorbic, glucoronic, maleic, fumaric, pyruvic, aspartic, glutamic, benzoic, anthranilic, mesylic, salicylic, p-hydroxybenzoic, phenylacetic, mandelic, ambonic, pamoic, methanesulfonic, ethanesulfonic, benzenesulfonic, pantothenic, 2-hydroxyethanesulfonic, toluenesulfonic, sulfanilic, cyclohexylaminosulfonic, stearic, algenic, β-hydroxybutyric, galactaric, and galacturonic acids. Suitable pharmaceutically acceptable base addition salts of compounds of formula I include metallic salts made from aluminum, calcium, lithium, magnesium, potassium, sodium, and zinc. Alternatively, organic salts made from N,N′-dibenzylethylenediamine, chloroprocaine, choline, diethanolamine, ethylenediamine, meglumine (N-methylglucamine) and procaine may be used form base addition salts of the compounds of formula I. All of these salts may be prepared by conventional means from the corresponding compounds of formula I by reacting, for example, the appropriate acid or base with the compound of formula I.

Also provided are pharmaceutical compositions for the prevention and/or treatment of undesirable, rapidly proliferating cells, such as for treating, preventing, or delaying the onset of a cancer in a subject in need of such treatment. The pharmaceutical composition comprises a therapeutically effective amount of a compound of formula I, or a derivative or pharmaceutically acceptable salt thereof, in association with at least one pharmaceutically acceptable carrier, adjuvant, or diluent (collectively referred to herein as “carrier materials”) and, if desired, other active ingredients. The active compounds of the present invention may be administered by any suitable route known to those skilled in the art, preferably in the form of a pharmaceutical composition adapted to such a route, and in a dose effective for the treatment intended. The active compounds and composition may, for example, be administered orally, intra-vascularly, intraperitoneally, intranasal, intrabronchial, subcutaneously, intramuscularly or topically (including aerosol). With some subjects local administration, rather than system administration, may be preferred. Formulation in a lipid vehicle may be used to enhance bioavailability.

The administration of the present invention may be for either prevention or treatment purposes. The methods and compositions used herein may be used alone or in conjunction with additional therapies known to those skilled in the art in the prevention or treatment of disorders characterized by unwanted, rapid proliferation of cells. Alternatively, the methods and compositions described herein may be used as adjunct therapy. By way of example, the apoptosis-inducing compounds of the present invention may be administered alone or in conjunction with other antineoplastic agents or other growth inhibiting agents or other drugs or nutrients.

There are large numbers of antineoplastic agents available in commercial use, in clinical evaluation and in pre-clinical development, which could be selected for treatment of cancers or other disorders characterized by rapid proliferation of cells by combination drug chemotherapy. Such antineoplastic agents fall into several major categories, namely, antibiotic-type agents, alkylating agents, antimetabolite agents, hormonal agents, immunological agents, interferon-type agents and a category of miscellaneous agents. Alternatively, other anti-neoplastic agents, such as metallomatrix proteases inhibitors (MMP), may be used. Suitable agents which may be used in combination therapy will be recognized by those of skill in the art. Similarly, when combination therapy is desired, radioprotective agents known to those of skill in the art may also be used.

The phrase “adjunct therapy” (or “combination therapy”), in defining use of a compound of the present invention and one or more other pharmaceutical agent, is intended to embrace administration of each agent in a sequential manner in a regimen that will provide beneficial effects of the drug combination, and is intended as well to embrace co-administration of these agents in a substantially simultaneous manner, such as in a single formulation having a fixed ratio of these active agents, or in multiple, separate formulations for each agent.

For oral administration, the pharmaceutical composition may be in the form of, for example, a tablet, capsule, suspension or liquid. The pharmaceutical composition is preferably made in the form of a dosage unit containing a particular amount of the active ingredient. Examples of such dosage units are capsules, tablets, powders, granules or a suspension, with conventional additives such as lactose, mannitol, corn starch or potato starch; with binders such as crystalline cellulose, cellulose derivatives, acacia, corn starch or gelatins; with disintegrators such as corn starch, potato starch or sodium carboxymethyl-cellulose; and with lubricants such as talc or magnesium stearate. The active ingredient may also be administered by injection as a composition wherein, for example, saline, dextrose or water may be used as a suitable carrier.

For intravenous, intramuscular, subcutaneous, or intraperitoneal administration, the compound may be combined with a sterile aqueous solution which is preferably isotonic with the blood of the recipient. Such formulations may be prepared by dissolving solid active ingredient in water containing physiologically compatible substances such as sodium chloride, glycine, and the like, and having a buffered pH compatible with physiological conditions to produce an aqueous solution, and rendering said solution sterile. The formulations may be present in unit or multi-dose containers such as sealed ampoules or vials.

If the unwanted proliferating cells are localized in the G.I. tract, the compound may be formulated with acid-stable, base-labile coatings known in the art which begin to dissolve in the high pH small intestine. Formulation to enhance local pharmacologic effects and reduce systemic uptake are preferred.

Formulations suitable for parenteral administration conveniently comprise a sterile aqueous preparation of the active compound which is preferably made isotonic. Preparations for injections may also be formulated by suspending or emulsifying the compounds in non-aqueous solvent, such as vegetable oil, synthetic aliphatic acid glycerides, esters of higher aliphatic acids or propylene glycol.

Formulations for topical use include known gels, creams, oils, and the like. For aerosol delivery, the compounds may be formulated with known aerosol exipients, such as saline, and administered using commercially available nebulizers. Formulation in a fatty acid source may be used to enhance biocompatibility.

For rectal administration, the active ingredient may be formulated into suppositories using bases which are solid at room temperature and melt or dissolve at body temperature. Commonly used bases include cocoa butter, glycerinated gelatin, hydrogenated vegetable oil, polyethylene glycols of various molecular weights, and fatty esters of polyethylene stearate.

The dosage form and amount can be readily established by reference to known treatment or prophylactic regiments. The amount of therapeutically active compound that is administered and the dosage regimen for treating a disease condition with the compounds and/or compositions of this invention depends on a variety of factors, including the age, weight, sex, and medical condition of the subject, the severity of the disease, the route and frequency of administration, and the particular compound employed, the location of the unwanted proliferating cells, as well as the pharmacokinetic properties of the individual treated, and thus may vary widely. The dosage will generally be lower if the compounds are administered locally rather than systemically, and for prevention rather than for treatment. Such treatments may be administered as often as necessary and for the period of time judged necessary by the treating physician. One of skill in the art will appreciate that the dosage regime or therapeutically effective amount of the inhibitor to be administrated may need to be optimized for each individual. The pharmaceutical compositions may contain active ingredient in the range of about 0.1 to 2000 mg, preferably in the range of about 0.5 to 500 mg and most preferably between about 1 and 200 mg. A daily dose of about 0.01 to 100 mg/kg body weight, preferably between about 0.1 and about 50 mg/kg body weight, may be appropriate. The daily dose can be administered in one to four doses per day.

### EXPERIMENTAL PROCEDURE

Cell culture—LNCaP androgen-dependent (p53+/+) and PC-3 androgen-nonresponsive (P53−/−) prostate cancer cells were obtained from the American Type Culture Collection (Manassas, Va.). The preparation of the stable Bcl-xL-overexpressing LNCaP clone B3 (LNCaP/B3) was previously described (18). PC-3, LNCaP, and LNCaP/B3 cells were maintained in RPMI 1640 supplemented with 10% fetal bovine serum (FBS) at 37° C. in a humidified incubator containing 5% carbon dioxide. Normal human prostate epithelial (PrEC) cells were purchased from Cambrex Bio Science Walkersville, Inc. (East Tutherford, N.J.). Cells were maintained in Prostate Epithelial Cell Medium with growth supplements at 37° C. in a humidified incubator containing 5% carbon dioxide. The recommended seeding density for subculture is 2,500 cells/cm². It takes 6-9 days from subculture to attain confluency.

Reagents—α-Tocopherol, α-tocopheryl succinate, 2,2,5,7,8-pentamethyl-6-chromanol and other chemical reagents required for the synthesis of various analogues were purchased from Aldrich Sigma (St. Louis, Mo.) unless otherwise indicated. Synthesis of TS-1 (succinic acid mono-[2-(4,8-dimethyl-nonyl)-2,5,7,8-tetramethyl-chroman-6-yl]ester), TS-2 (succinic acid mono-[2,5,7,8-tetramethyl-2-(4-methyl-pentyl)-chroman-6-yl]ester), TS-3 (succinic acid mono-(2,2,5,7,8-pentamethyl-chroman-6-yl) ester), TS-4 (2-(4,8-dimethyl-nonyl)-2,5,7,8-tetramethyl-chroman-6-ol), and TS-5 (3-[2,5,7,8-tetramethyl-2-(4,8-dimethyl-nonyl)-chroman-6-yloxy]propionic acid) will be published elsewhere. The identity, purity (9%) of these synthetic derivatives were verified by proton nuclear magnetic resonance, high resolution mass spectrometry, and elemental analysis. MTT [3-(4,5-dimethylthiazol-2-yl)-2,5-diphenyl-2H-tetrazolium bromide)] for cell viability assay were purchased form TCI America, Inc. (Portland, Oreg.). The Cell Death Detection ELISA kit was purchased from Roche Diagnostics (Mannheim, Germany). Rabbit antibodies against Bcl-xL, Bax, Bak, Bid, PARP, and cleaved caspases-9 were purchased from Cell Signaling Technology, Inc. (Beverly, Mass.). Rabbit antibodies against Bad, cytochrome c, and mouse anti-Bcl-2 were from Santa Cruz Biotechnology, Inc. (Santa Cruz, Calif.). Mouse monoclonal anti-actin was from ICN Biomedicals, Inc. (Costa Mesa, Calif.).

Cell viability analysis—The effect of individual test agents on cell viability was assessed by using the MTT assay in 6 to 12 replicates. PC3, LNCaP, and B3-LNCaP cells were seeded and incubated in poly-p-lysine-coated, 96-well, flat-bottomed plates in RPMI 1640 medium supplemented with 10% FBS medium for 24 hours. PrEC cells were seeded at the recommend density in 96-well, flat-bottomed plates in Prostate Epithelial Cell Medium with growth supplements for 3 days. All cells were exposed to various concentrations of test agents dissolved in ethanol (for α-tocopherol, α-tocopheryl succinate, and TS-3) or DMSO (all other test agents used) with a final concentration of 0.1% in serum-free RPMI 1640 medium for PC3, LNCaP, and LNCaP/B3 cells or in Prostate Epithelial Cell Basal Medium with growth supplements for PrEC cells. Controls received DMSO or ethanol vehicle at a concentration equal to that in drug-treated cells. The medium was removed, replaced by 200 μL of 0.5 mM MTT in 10% FBS-containing RPMI 1640 medium, and cells were incubated in the CO2 incubator at 37° C. for 2 h. Supernatants were removed from the wells and the reduced MTT dye was solubilized in 200 μL/well of DMSO. Absorbance at 570 nm was determined on a plate reader.

Apoptosis detection by ELISA—Induction of apoptosis was assessed with a Cell Death Detection ELISA kit (Roche Diagnostics) by following the manufacturer's instruction. This test is based on the quantitative determination of cytoplasmic histone-associated DNA fragments in the form of mononucleosomes and oligonucleosomes after induced apoptotic death. In brief, 5×106 cells were cultured in a T-25 flask in 10% FBS-containing medium for 24 h, and were treated with the test agents at various concentrations in serum-free medium for 24 hours. Both floating and adherent cells were collected; cell lysates equivalent to 5×105 cells were used in the ELISA.

Western blot analysis of cytochrome c release into the cytoplasm—Cytosolic-specific, mitochondria-free lysates were prepared according to an established procedure (18). In brief, after individual treatments for 24 h, both the incubation medium and adherent cells in T-75 flasks were collected and centrifuged at 600×g for 5 min. The pellet fraction was recovered, placed on ice, and triturated with 100 μL of a chilled hypotonic lysis solution [50 mM PIPES-KOH (pH 7.4) containing 220 mM mannitol, 68 mM sucrose, 50 mM KCl, 5 mM EDTA, 2 mM MgCl₂, 1 mM dithiothreitol, and a mixture of protease inhibitors including 100 μM 4-(2-aminoethyl)benzenesulfonyl fluoride, 80 nM aprotinin, 5 μM bestatin, 1.5 μM E-64 protease inhibitor, 2 μM leupeptin, and 1 μM. pepstatin A]. After a 45-min incubation on ice, the mixture was centrifuged at 600×g for 10 min. The supernatant was collected in a microcentrifuge tube, and centrifuged at 14,000 rpm for 30 min. An equivalent amount of protein (50 μg) from each supernatant was resolved in 15% SDS-polyacrylamide gel. Bands were transferred to nitrocellulose membranes and analyzed by immunoblotting with anti-cytochrome c antibodies as described below.

Immunoblotting—Cells were seeded in 10% FBS-containing RPMI-1640 medium for 24 h and treated with various agents as aforementioned. After individual treatments for 24 h, the adherent cells in T-25 or T-75 flasks were scraped, combined with the medium, and centrifuged at 2200 rpm for 10 min. The supernatants were recovered, placed on ice, and triturated with 20 to 50 μL of a chilled lysis buffer (M-PER Mammalian Protein Extraction Reagent; Pierce, Rockford, Ill.), to which was added 1% protease inhibitor cocktail (set III; EMD Biosciences, Inc., San Diego, Calif.). After a 30-min incubation on ice, the mixture was centrifuged at 16,100×g for 3 min. Two μL of the suspension was taken for protein analysis using the Bradford assay kit (Bio-Rad, Hercules, Calif.); to the remaining solution was added the same volume of 2×SDS-polyacryl-amide gel electrophoresis sample loading buffer (100 mM Tris-HCl, pH 6.8, 4% SDS, 5% β-mercaptoethanol, 20% glycerol, and 0.1% bromphenol blue). The mixture was boiled for 10 min. Equal amounts of proteins were loaded onto 8-12% SDS-polyacrylamide gels. After electrophoresis, protein bands were transferred to nitrocellulose membranes in a semidry transfer cell. The transblotted membrane was blocked with Tris-buffered saline/0.1% Tween 20 (TBST) containing 5% nonfat milk for 90 min, and the membrane was incubated with the appropriate primary antibody in TBST/5% nonfat milk at 4° C. overnight. After washing three times with TBST for a total of 45 min, the transblotted membrane was incubated with goat anti-rabbit or anti-mouse IgG-horseradish peroxidase conjugates (diluted 1:1000) for 1 h at room temperature and washed four times with TBST for a total of 1 h. The immunoblots were visualized by enhanced chemiluminescence.

Competitive fluorescence polarization assay—The binding affinity of the test agent to Bcl-XL was analyzed by a competitive fluorescence polarization assay in which the ability of the agent to displace the binding of a Bak BH3-domain peptide to either Bcl-2 or Bcl-XL was determined. Flu-BakBH3, a Bak-BH3 peptide labeled at the NH₂ terminus with fluorescein, was purchased from Genemed Synthesis (San Francisco, Calif.). COOH-terminal-truncated, His-tagged Bcl-XL was purchased from EMD Biosciences (San Diego, Calif.) and soluble glutathione S-transferase-fused Bcl-2 was obtained from Santa Cruz Biotechnology. The binding analysis was carried out in a dual-path length quartz cell with readings taken at λem 480 nm and λex 530 nm at room temperature using a luminescence spectrometer according to an established procedure (19).

Determination of 1050 values—Data from cell viability and fluorescence polarization assays were analyzed by using the CalcuSyn software (Biosoft, Ferguson, Mo.) to determine IC50 values, in which the calculation was based on the medium-effect equation [i.e., log(fa/fu)=m log(D)−m log(Dm), where fa and fu denote fraction affected and unaffected, respectively; in represents the Hill-type coefficient signifying the sigmoidicity of the dose-effect curve; and D and Dm are the dose used and IC50, respectively.

Co-immunoprecipitation—PC3 cells treated with 40 μM α-tocopheryl succinate or 10 μM TS-1 for 24 h were scraped off the flask, transferred into centrifuge tubes, and centrifuged at 2200 rpm for 10 min to pellet the cells. The pellet was resuspended in ice-cold 0.5 mL of radioimmunoprecipitation assay buffer (50 mM Tris-HCl, pH 7.4, 1% Nonidet P-40, 0.25% sodium deoxycholate, 150 mM NaCl, 1 mM EDTA, and 1% protease inhibitor cocktail) and gently mixed on an orbital shaker at 4° C. for 15 min, followed by centrifugation at 14,000×g for 15 min to yield cell lysates. These cell lysates were treated with 100 μL of protein A-agarose bead slurry followed by brief centrifugation to remove nonspecific binding proteins. Equal amounts of proteins from these lysates, as determined by the Bradford assay, were mixed with anti-Bcl-2 or anti Bcl-XL antibodies in an orbital shaker at 23° C. for 2 h, followed by 100 μL of protein A-agarose head slurry at 4° C. for 12 h. The immunocomplex was collected by brief centrifugation, washed four times with 800 μL of ice-cold radioimmunoprecipitation assay buffer, and suspended in 50 μL of 2×SDS sample loading buffer. The suspension was boiled for 10 min, cooled, and briefly centrifuged to remove the heads. Western blot analysis with antibodies against Bak as described above.

Molecular modeling—Human Bcl-xL crystal structure, obtained from the Brookhaven Protein Data bank (entry code of 1R2D) (20) was subject to the deletion of water molecules, the addition of all hydrogens, and the assignment of Gasteiger charges (21), and then non-polar hydrogens were merged. 3-D affinity grids centered on the Bak peptide binding site with 0.375 Å spacing were calculated for each of the following atom types: a) protein: A (aromatic C), C, HD, N, NA, OA, SA; b) ligands: C, A, N, NA, OA, S, SA, HD, Br, e (electrostatic) and d (desolvation) using Autogrid4. AutoDock version 4.0.0 was used for the docking simulation. We selected the Lamarckian genetic algorithm (LGA) for ligand conformational searching because it has enhanced performance relative to simulated annealing or the simple genetic algorithm. The ligand's translation, rotation and internal torsions are defined as its state variables and each gene represents a state variable. LGA adds local minimization to the genetic algorithm, enabling modification of the gene population. For each compound, the docking parameters were as follows: trials of 100 dockings, population size of 150, random starting position and conformation, translation step ranges of 2.0 Å, rotation step ranges of 50°, elitism of 1, mutation rate of 0.02, crossover rate of 0.8, local search rate of 0.06, and 100 million energy evaluations. Final docked conformations were clustered using a tolerance of 2.0 Å root-mean-square deviation (RMSD).

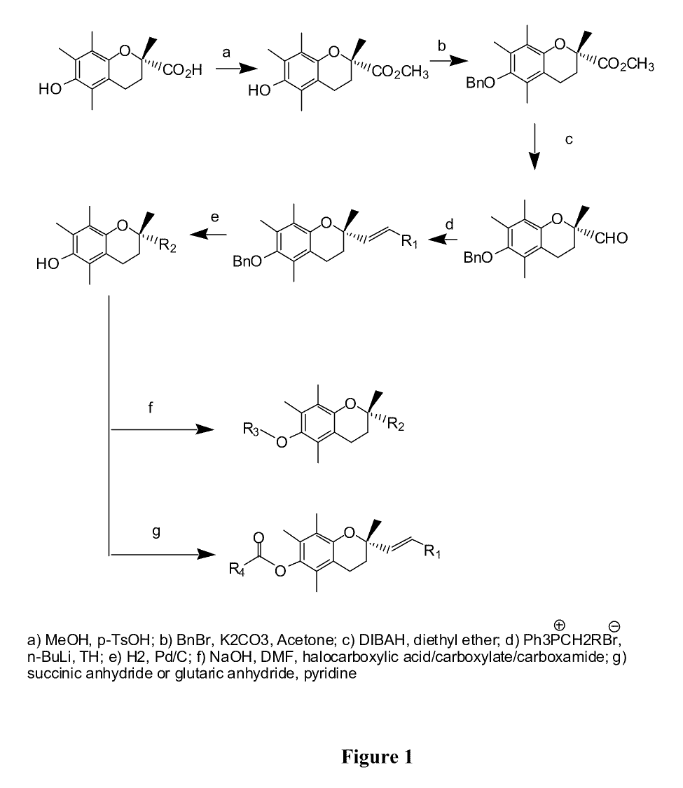

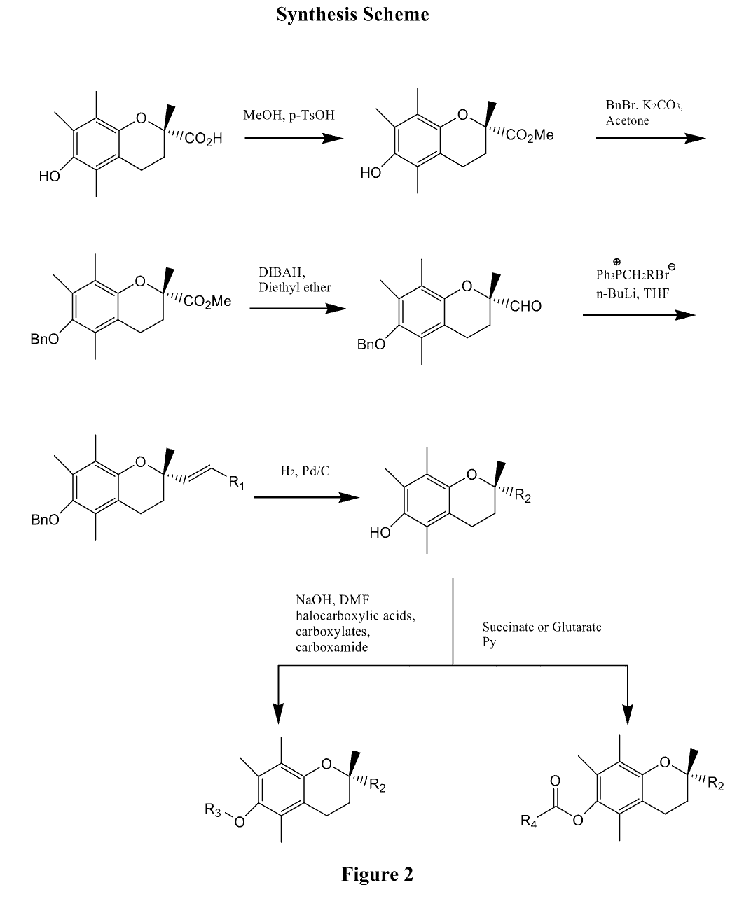

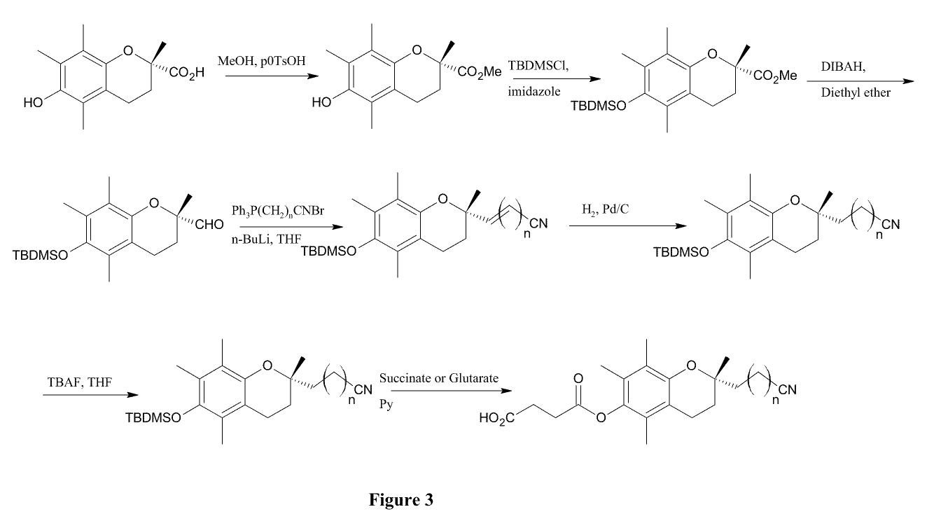

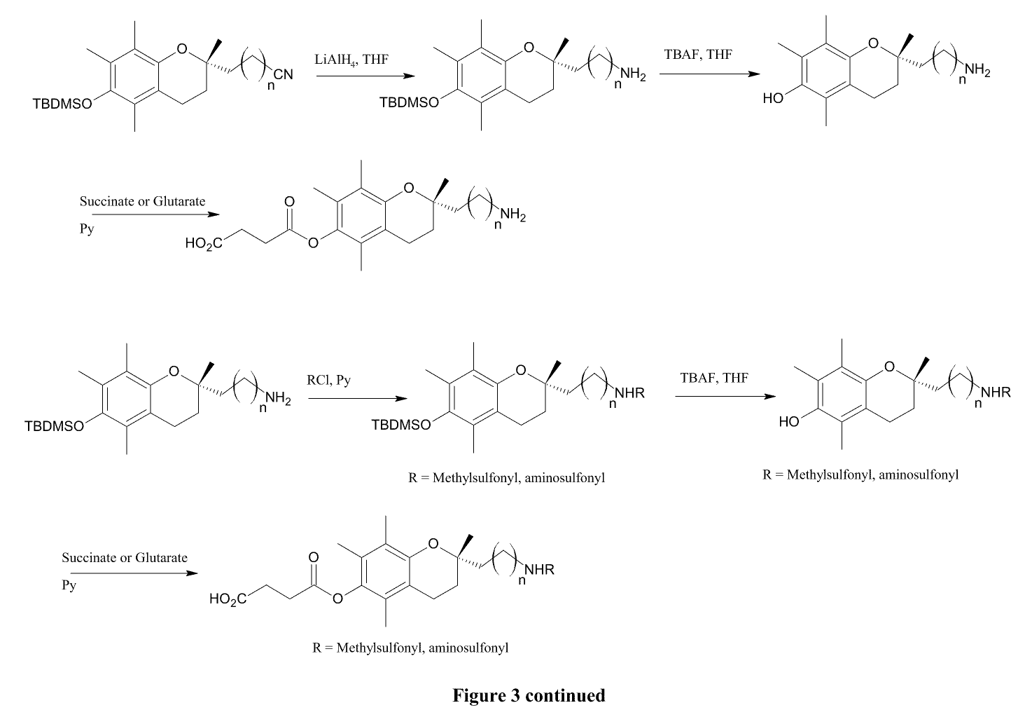

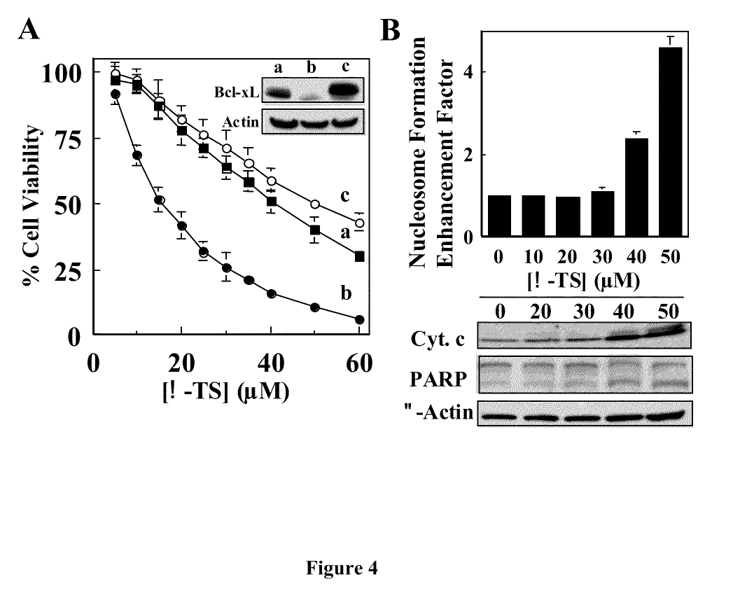

Referring now to the figures, FIG. 1 shows the synthetic scheme for preparing the compounds described herein. FIG. 4 shows differential sensitivity of PC-3, LNCaP, and Bcl-xL-overexpressing LNCaP (LNCaP/B3) cells to α-tocopheryl succinate-induced apoptosis. (A) Dose-dependent effects of α-tocopheryl succinate on the viability of PC-3, LNCaP, and LNCaP/B3 cells after 24-h exposure in serum-free RPMI 1640 medium. Points, mean; bars, SD (n=6). (B) Evidence of apoptotic death in α-tocopheryl succinate-treated PC-3 cells. Upper panel, formation of nucleosomal DNA in PC-3 cells that were treated with α-tocopheryl succinate at the indicated concentrations for 24 h. DNA fragmentation was quantitatively measured by a cell death detection ELISA kit. Columns, mean; bars, SD (n=3). Lower panel, cytochrome c release into cytoplasm and PARP cleavage induced by different doses of α-tocopheryl succinate in PC-3 cells. PC-3 cells were treated with the drug at the indicated doses for 24 h in serum-free RPMI 1640 medium. Equivalent amounts of proteins from mitochondrial-free cell lysates were electrophoresed and probed by Western blotting with the respective antibodies.

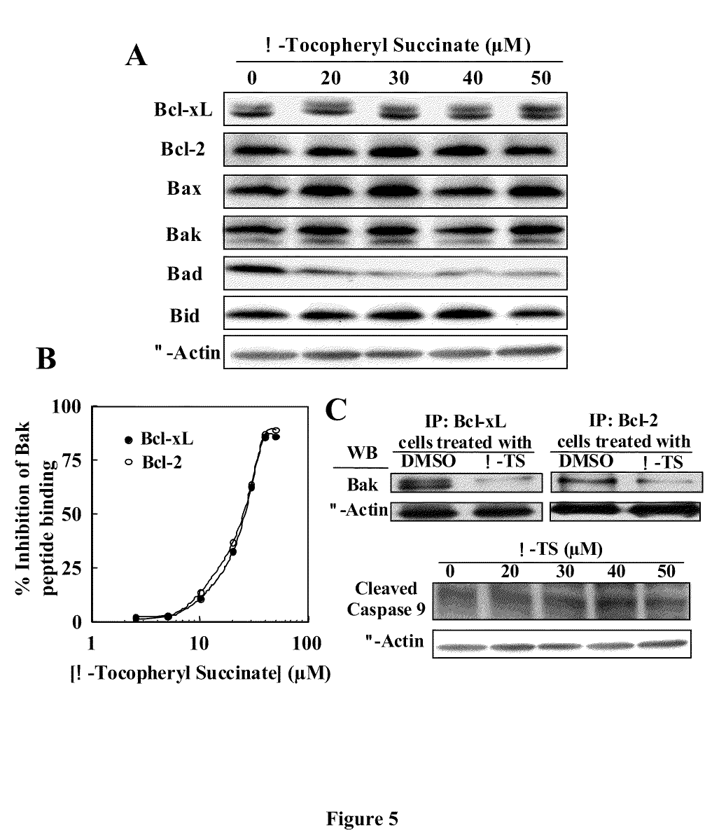

FIG. 5 shows α-Tocopheryl succinate blocks Bcl-xL/Bcl-2 function by inhibiting BH3 domain-mediated heterodimerization. (A) α-Tocopheryl succinate has no apparent effect on the expression levels of Bcl-2 family members, except Bad, in PC-3 cells. PC-3 cells were exposed different doses of α-tocopheryl succinate in serum-free RPMI 1640 medium for 24. Equivalent amounts of proteins from cell lysates were electrophoresed and probed by Western blotting with individual antibodies. (B) Dose-dependent inhibition of BH3 domain-mediated protein interactions of Bak BH3 peptide with Bcl-xL and Bcl-2 by α-tocopheryl succinate. The curve represents the displacement of Flu-BakBH3 peptide from Bcl-xL or Bcl-2 by α-tocopheryl succinate at the indicated concentrations, as described in the Experimental Procedures. (C) α-Tocopheryl succinate triggers caspase-dependent apoptotic death by inhibiting heterodimer formation of Bcl-xL and Bcl-2 with Bak. Upper panel, effect of α-tocopheryl succinate on the dynamics of Bcl-xL/Bak (left) and Bcl-2/Bak (right) interactions in PC-3 cells. PC-3 cells were exposed to 40 μM α-tocopheryl succinate or DMSO vehicle for 12 h, and cell lysates were immunoprecipitated (IP) with anti-Bcl-xL or anti-Bcl-2 antibodies. The immunoprecipitates were probed with anti-Bak antibodies by Western blot analysis (WB). Lower panel, Dose-dependent effect of α-tocopheryl succinate on caspase-9 activation in PC-3 cells. PC-3 cells were treated with α-tocopheryl succinate at the indicated concentrations for 24 h. Caspase-9 antibodies recognize the large subunits (39 and 37 kDa).

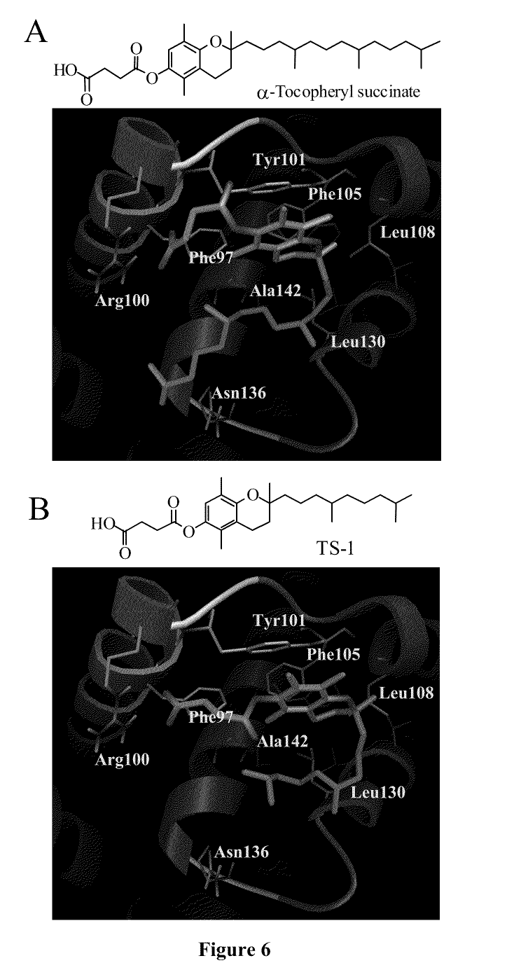

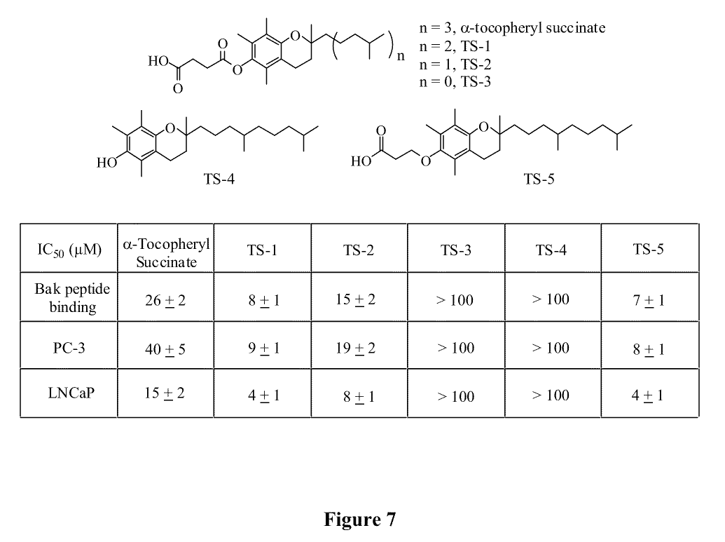

FIG. 6 shows modeled docking of α-tocopheryl succinate (upper panel) and TS-1 into the Bak BH3 peptide-binding site of Bcl-xL. FIG. 7 shows structures and potency for inhibiting Bak BH3 peptide binding to Bcl-xL and for suppressing the viability of PC-3 and LNCaP cells for α-tocopheryl succinate and TS-1-TS-5. The general structure of α-tocopheryl succinate and TS-1-TS-3 and structures of TS-4 and TS-5 are shown at the top. N represents the number of the isopranyl units in the aliphatic side chain. The reported IC₅₀ values are concentrations at which Bak BH3 peptide binding is inhibited by 50% or at which PC-3 or LNCaP cell death measures 50% relative to DMSO control after 24 h-exposure in serum-free RPMI 1640 medium.

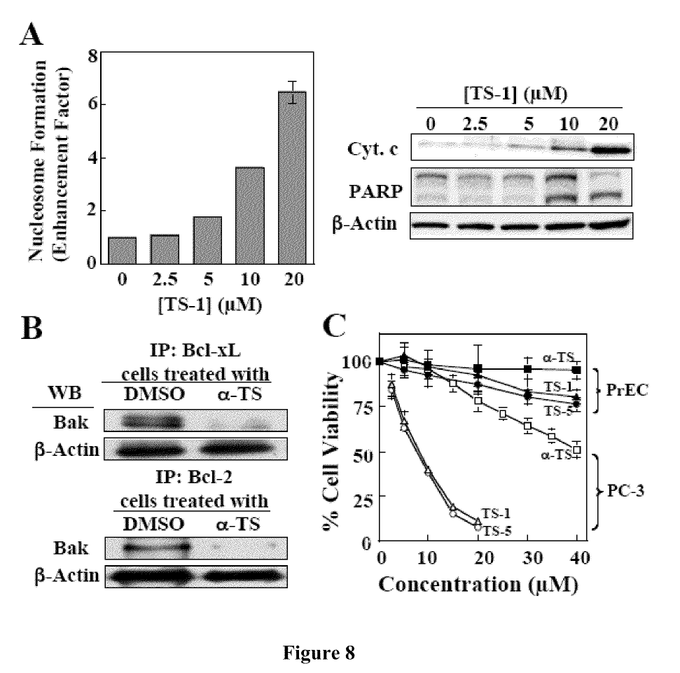

FIG. 8 shows mechanistic validation of the antitumor action of TS-1. (A) Evidence of apoptotic death in drug-treated PC-3 cells. Left, formation of cytoplasmic nucleosomal DNA in drug-treated PC-3 cells at the indicated concentrations. DNA fragmentation was quantitatively measured by a cell death detection ELISA kit. Columns, mean; bars, SD (n=3). Right, cytochrome c release into cytoplasm and PARP cleavage induced by different doses of TS-1 in PC-3 cells. PC-3 cells were treated with the drug at the indicated doses for 24 h in serum-free RPMI 1640 medium. Equivalent amounts of proteins from mitochondrial-free cell lysates were electrophoresed and probed by Western blotting with the respective antibodies. (B) Effect of TS-1 on the dynamics of Bcl-xL/Bak (left) and Bcl-2/Bak (right) interactions in PC-3 cells. PC-3 cells were exposed to 20 μM TS-1 or DMSO vehicle for 12 h, and cell lysates were immunoprecipitated (IP) with anti-Bcl-xL or anti-Bcl-2 antibodies. The immunoprecipitates were probed with anti-Bak antibodies by Western blot analysis (WB). (C) Dose-dependent effect of α-tocopheryl succinate, TS-1, and TS-5 on the viability of PrECs. Cells were exposed to the indicated concentrations of the test agent in Prostate Epithelial Cell Basal Medium with growth supplements for 24 h. Control PC-3 cells received DMSO or ethanol vehicles. Cell viability was analyzed by MTT assay.

Differential susceptibility of LNCaP and PC-3 prostate cancer cell lines to α-tocopheryl succinate As part of our effort to understand the mode of action of α-tocopheryl succinate, we examined its antiproliferative effect in two human prostate cancer cell lines, LNCaP and PC-3. Of these two types of cells, LNCaP cells were more susceptible to the proliferation inhibition than PC-3 cells, with IC₅₀ values of 15 μM and 40 respectively (FIG. 4A). This reduction in cell viability was, at least in part, attributable to mitochondria-dependent apoptosis induction, as evidenced by DNA fragmentation, cytochrome c release, and PARP cleavage (FIG. 4B). As both LNCaP and PC-3 cells exhibit up-regulated phosphatidylinositol 3-kinase (PI3K)/Akt signaling due to loss of PTEN function, this differential sensitivity might be attributable to differences in the respective ability to maintain mitochondrial integrity in response to apoptotic signals. Data from this and other laboratories have demonstrated that PC-3 cells were resistant to the apoptosis-inducing effect of many therapeutic agents due to Bcl-xL overexpression.

Ectopic Bcl-xL expression protects LNCaP cells from α-tocopheryl succinate-induced apoptosis To examine the possibility that the high expression level of Bcl-xL PC-3 cells might underlie the resistance, we assessed the effect of enforced Bcl-xL expression in a stably transfected LNCaP clone (LNCaP/B3) on α-tocopheryl succinate-induced cell death. The expression level of ectopic Bcl-xL in B3 cells was approximately fivefold of that of the endogenous counterpart in PC-3 cells (FIG. 4A, inset), while that of Bcl-2 was slightly lower in the LNCaP/B3 cells. The high level of ectopic Bcl-xL expression in LNCaP/B3 cells substantially increased the resistance of LNCaP cells to α-tocopheryl succinate-induced cell death, to a degree greater than that of PC-3 cells (FIG. 4A).

α-Tocopheryl succinate is an inhibitor of Bcl-xL function The above finding suggested that α-tocopheryl succinate-mediated apoptosis might involve the modulation of the function of Bcl-xL and/or other Bcl-2 members. Accordingly, we examined this putative link at both transcriptional and posttranscriptional levels. First, we assessed the dose-dependent effect of α-tocopheryl succinate on the expression of different Bcl-2 family members in PC-3 cells, including Bcl-xL, Bcl-2, Bax, Bak, Bad, and Bid by Western blotting. FIG. 5A indicates that with the exception of a decrease in Bad expression, the exposure to α-tocopheryl succinate did not cause appreciable changes in the expression level of these Bcl-2 members. Second, we used a competitive fluorescence polarization analysis to investigate the effect of α-tocopheryl succinate on the binding of a fluorescein-labeled Bak BH3 domain peptide to Bcl-xL and Bcl-2. FIG. 5B depicts the ability of α-tocopheryl succinate to disrupt the BH3 domain-mediated interactions with Bcl-xL, and Bcl-2 with equal potency, with IC₅₀ of 26±2 μM.

To confirm the mode of action of α-tocopheryl succinate, we assessed the intracellular effects on the dynamics of Bcl-xL/Bak and Bcl-2/Bak interactions in PC-3 cells. Lysates from PC-3 cells treated with α-tocopheryl succinate vis-à-vis DMSO vehicle for 12 h were immunoprecipitated with antibodies against Bcl-xL or Bcl-2. Probing of the immunoprecipitates with anti-Bak antibodies by Western blotting indicates that the level of Bak associated with Bcl-xL and Bcl-2 was significantly reduced compared with the DMSO control (FIG. 5C, upper panel). This decrease in intracellular association bore out the above in vitro binding data. As Bcl-xL and Bcl-2 abrogated the effects of Bak and other proapoptotic Bcl-2 members through BH3 domain-mediated heterodimerization, we also showed that this decrease in Bak binding was accompanied by caspase-9 activation in a dose-dependent manner in drug-treated cells (FIG. 5C, lower panel).

Together, these data demonstrate that the effect of α-tocopheryl succinate on apoptosis in prostate cancer cells was, at least in part, mediated through the inhibition of Bcl-xL function by disrupting BH3 domain-mediated hetero-dimerization. From a translational perspective, this mechanistic finding provided a molecular basis to structurally optimize this agent to develop potent Bcl-xL/Bcl-2 binding inhibitors.

Molecular docking of α-tocopheryl succinate into the Bak peptide-binding site of Bcl-xL α-Tocopheryl succinate was docked into the Bak peptide-binding site that is located in a hydrophobic cleft bound by the BH1, BH2, and BH3 regions of Bcl-xL. Docking analysis indicates that α-tocopheryl succinate adopted a unique hairpin-shaped conformation in interacting with this hydrophobic pocket (FIG. 6A). As shown, the carboxylic terminus of the hemisuccinate formed electrostatic interactions and hydrogen bonding with the guanidino side chain of Arg100. While the chroman aromatic ring interacted with Tyr101 and Phe105 through π-π interactions, the phytyl chain coiled back to gain access to the hydrophobic side chain of Leu108, Leu130, and Ala142. However, the terminal isopranyl unit of the aliphatic long chain overhanged into a polar region that consisted of Asn136, the amide backbone of Trp 137, Gly138, and Arg130 located at the beginning end of a large helical dipole, and solvent.

α-Tocopheryl succinate derivatives with truncated side chains exhibit higher potency in Bcl-xL inhibition. This computer model shed light onto the mode of binding of α-tocopheryl succinate to Bcl-xL, and provided a molecular basis for structural optimization. We rationalized that the hemisuccinate and the two proximal isopranyl units of the side chain play a crucial role in ligand anchoring and stabilization of the protein-ligand complex, respectively. However, exposure of the distal isopranyl unit to a polar environment might diminish the binding affinity of α-tocopheryl succinate. This premise was corroborated by docking TS-1, an analogue with one isopranyl unit removed from the phytyl side chain, into the Bcl-xL binding domain (FIG. 6B). The mode of binding of this truncated analogues was analogous to that of α-tocopheryl succinate, however, without the unfavorable interaction with the polar milieu. Theoretical ΔG_{binding} values were calculated to be −7.5 kcal/mol and −8.1 kcal/mol for α-tocopheryl succinate and TS-1, respectively, of which the discrepancy would give rise to a 3-fold difference in binding affinity.

To validate the above modeling data, we carried out structural modifications of a-tocopheryl succinate by gradually removing the isopranyl unit from its phytyl side chain, yielding TS-1, TS-2, and TS-3 (FIG. 7A). In addition, TS-4 and TS-5 were synthesized to verify the role of the terminal carboxylic function in ligand anchoring, which represented TS-1 analogues with the hemisuccinate removed and replaced with an ether-linked propionate, respectively.

Functional assays indicate that the potency of these derivatives vis-à-vis α-tocopheryl succinate in inhibiting Bak peptide-Bcl-xL binding paralleled that of suppressing cell viability (FIG. 7B). The potency was in the order of TS-1, TS-5>TS-2>α-tocopheryl succinate, while TS-3 and TS-4 lacked appreciable activity even at 100 μM. There existed a 3-fold difference in IC₅₀ between TS-1 and α-tocopheryl succinate in blocking Bak peptide binding to Bcl-xL, which is consistent with that of the theoretical calculation.

The differential inhibitory activity among α-tocopheryl succinate and its truncated analogues underlies the subtle impact of the length of the hydrophobic side chain on Bcl-xL binding. However, complete removal of the side chain in TS-3 abrogated the binding affinity, supporting the role of this hydrophobic interaction in the stabilization of protein-ligand complexes. In addition, replacement of the succinate with an ether-linked propionate had no effect on the Bcl-xL-binding and antiproliferative activities. This finding suggests that the carbonyl group of the hemisuccinate ester linkage was not involved in ligand binding, consistent with the modeled recognition mode (FIG. 6).

Evidence indicates that TS-1 mediated antiproliferative effects in PC-3 cells through the same mechanism as α-tocopheryl succinate. TS-1-induced apoptotic death was evidenced by DNA fragmentation, cytochrome c release, and PARP cleavage (FIG. 8A). Moreover, 10 μM TG-1 abolished the intracellular binding of Bak to Bcl-xL, and Bcl-2 in PC-3 cells (FIG. 8B). In contrast to LNCaP and PC-3 cells, normal prostate epithelial cells (PrECs) were resistant to the antiproliferative effect of TS-1 and TS-5, similar to that observed with α-tocopheryl succinate (FIG. 8C). This differential sensitivity indicates that the effect of TS-1 and TS-3 on apoptosis was tumor cell-specific.

The examples described herein are for illustrative purposes only and are not meant to limit the scope of the claimed invention.

1-19. (canceled)

20. A compound of formula I: 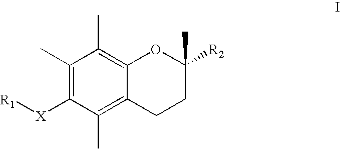
wherein X is selected from the group consisting of oxygen, nitrogen and sulfur;
R₁ is selected from the group consisting of carboxylic acids, carboxylates, carboxamides, and carboxylic acid esters; and
R₂ is an alkyl group including from 6 to 11 carbon atoms;
or a pharmaceutically acceptable salt thereof.

21. The compound of claim 20, wherein X is O.

22. The compound of claim 20, wherein R₂ includes 1 or 2 isopranyl units.

23. The compound of claim 20, wherein R₁ is a carboxylic acid or carboxylic acid ester including from 2 to 5 carbon atoms.

24. The compound of claim 21, wherein R₂ includes 1 or 2 isopranyl units and R₁ is a carboxylic acid or carboxylic acid ester including from 2 to 5 carbon atoms.

25. The compound of claim 20, wherein the compound is selected from the group consisting of 3-[2,5,7,8-tetramethyl-2-(4-methyl-pentyl)-chroman-6-yloxy]-acetic acid; 3-[2,5,7,8-tetramethyl-2-(4-methyl-pentyl)-chroman-6-yloxy]-propionic acid; 3-[2,5,7,8-tetramethyl-2-(4-methyl-pentyl)-chroman-6-yloxy]-butyric acid; [2-(4,8-dimethyl-nonyl)-2,5,7,8-tetramethylchroman-6-yloxy]-acetic acid; 3-[2-(4,8-dimethyl-nonyl)-2,5,7,8-tetramethylchroman-6-yloxy]-propionic acid; 4-[2-(4,8-dimethyl-nonyl)-2,5,7,8-tetramethylchroman-6-yloxy]-butyric acid; succinic acid mono-[2,5,7,8-tetramethyl-2-(4-methyl-pentyl)-chroman-6-yl]ester; pentanedioic acid mono-[2,5,7,8-tetramethyl-2-(4-methyl-pentyl)-chroman-6-yl]ester; succinic acid mono-[2-(4,8-dimethyl-nonyl)-2,5,7,8-tetramethylchroman-6-yl]ester; and pentanedioic acid mono-[2-(4,8-dimethyl-nonyl)-2,5,7,8-tetramethylchroman-6-yl]ester; or a pharmaceutically acceptable salt thereof.

26. The compound of claim 20, wherein the compound is selected from the group consisting of succinic acid mono-[2-(4,8-dimethyl-nonyl)-2,5,7,8-tetramethylchroman-6-yl]ester; succinic acid mono-[2,5,7,8-tetramethyl-2-(4-methyl-pentyl)-chroman-6-yl]ester; and 3-[2-(4,8-dimethyl-nonyl)-2,5,7,8-tetramethylchroman-6-yloxy]-propionic acid, or a pharmaceutically acceptable salt thereof.

27. A method for the treatment of a cell proliferative disease in a subject, comprising administering to a pharmacologically effective dose of compound I 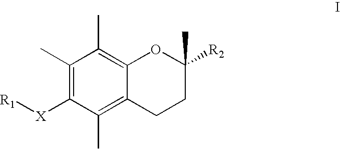
wherein X is selected from the group consisting of oxygen, nitrogen and sulfur;
R₁ is selected from the group consisting of carboxylic acids, carboxylates, carboxamides, and carboxylic acid esters; and
R₂ is an alkyl group including from 6 to 11 carbon atoms;
or a pharmaceutically acceptable salt thereof.

28. The method of claim 27, wherein X is O.

29. The method of claim 27, wherein R₂ includes 1 or 2 isopranyl units.

30. The method of claim 27, wherein R₁ is a carboxylic acid or carboxylic acid ester including from 2 to 5 carbon atoms.

31. The method of claim 28, wherein R₂ includes 1 or 2 isopranyl units and R₁ is a carboxylic acid or carboxylic acid ester including from 2 to 5 carbon atoms.

32. The method of claim 27, wherein the compound is selected from the group consisting of 3-[2,5,7,8-tetramethyl-2-(4-methyl-pentyl)-chroman-6-yloxy]-acetic acid; 3-[2,5,7,8-tetramethyl-2-(4-methyl-pentyl)-chroman-6-yloxy]-propionic acid; 3-[2,5,7,8-tetramethyl-2-(4-methyl-pentyl)-chroman-6-yloxy]-butyric acid; [2-(4,8-dimethyl-nonyl)-2,5,7,8-tetramethylchroman-6-yloxy]-acetic acid; 3-[2-(4,8-dimethyl-nonyl)-2,5,7,8-tetramethylchroman-6-yloxy]-propionic acid; 4-[2-(4,8-dimethyl-nonyl)-2,5,7,8-tetramethylchroman-6-yloxy]-butyric acid; succinic acid mono-[2,5,7,8-tetramethyl-2-(4-methyl-pentyl)-chroman-6-yl]ester; pentanedioic acid mono-[2,5,7,8-tetramethyl-2-(4-methyl-pentyl)-chroman-6-yl]ester; succinic acid mono-[2-(4,8-dimethyl-nonyl)-2,5,7,8-tetramethylchroman-6-yl]ester; and pentanedioic acid mono-[2-(4,8-dimethyl-nonyl)-2,5,7,8-tetramethylchroman-6-yl]ester; or a pharmaceutically acceptable salt thereof.

33. The method of claim 27, wherein the compound is selected from the group consisting of succinic acid mono-[2-(4,8-dimethyl-nonyl)-2,5,7,8-tetramethylchroman-6-yl]ester; succinic acid mono-[2,5,7,8-tetramethyl-2-(4-methyl-pentyl)-chroman-6-yl]ester; and 3-[2-(4,8-dimethyl-nonyl)-2,5,7,8-tetramethylchroman-6-yloxy]-propionic acid, or a pharmaceutically acceptable salt thereof.

34. The method of claim 27, wherein the compound exhibits an anti-proliferative effect comprising apoptosis, cell cycle arrest, cellular differentiation, or DNA synthesis arrest.

35. A pharmaceutical composition, comprising one or more compounds of formula I: 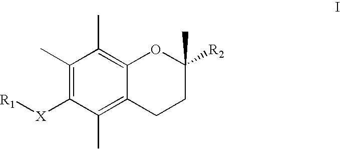
wherein X is selected from the group consisting of oxygen, nitrogen and sulfur;
R₁ is selected from the group consisting of carboxylic acids, carboxylates, carboxamides, and carboxylic acid esters; and
R₂ is an alkyl group including from 6 to 11 carbon atoms;
or a pharmaceutically acceptable salt thereof.

36. The pharmaceutical composition of claim 35, wherein X is O.

37. The pharmaceutical composition of claim 35 further comprising one or more adjuvants.

38. The pharmaceutical composition of claim 35 further comprising one or more diluents.

39. The pharmaceutical composition of claim 35, wherein the one or more compounds are selected from the group consisting of 3-[2,5,7,8-tetramethyl-2-(4-methyl-pentyl)-chroman-6-yloxy]-acetic acid; 3-[2,5,7,8-tetramethyl-2-(4-methyl-pentyl)-chroman-6-yloxy]-propionic acid; 3-[2,5,7,8-tetramethyl-2-(4-methyl-pentyl)-chroman-6-yloxy]-butyric acid; [2-(4,8-dimethyl-nonyl)-2,5,7,8-tetramethylchroman-6-yloxy]-acetic acid; 3-[2-(4,8-dimethyl-nonyl)-2,5,7,8-tetramethylchroman-6-yloxy]-propionic acid; 4-[2-(4,8-dimethyl-nonyl)-2,5,7,8-tetramethylchroman-6-yloxy]-butyric acid; succinic acid mono-[2,5,7,8-tetramethyl-2-(4-methyl-pentyl)-chroman-6-yl]ester; pentanedioic acid mono-[2,5,7,8-tetramethyl-2-(4-methyl-pentyl)-chroman-6-yl]ester; succinic acid mono-[2-(4,8-dimethyl-nonyl)-2,5,7,8-tetramethylchroman-6-yl]ester; and pentanedioic acid mono-[2-(4,8-dimethyl-nonyl)-2,5,7,8-tetramethylchroman-6-yl]ester; or a pharmaceutically acceptable salt thereof.
